(19) United States
(12) Patent Application Publication
(10) Pub. No.: US 2006/0064005 A1
(43) Pub. Date: Mar. 23, 2006

(54) System and method for externally controlled surgical navigation

(75) Inventors: John J. Triano, Richardson, TX (US); Carolyn Rogers, Frisco, TX (US); Dennis Colleran, Frisco, TX (US); Michael S. Hisey, Flower Mound, TX (US); Scott Schorer, Longmont, CO (US)

Correspondence Address: DALLAS OFFICE OF FULBRIGHT & JAWORSKI L.L.P., 2200 ROSS AVENUE, SUITE 2800, DALLAS, TX, 75201-2784, US

(73) Assignee: Innovative Spinal Technologies, Plano, TX (US)

(21) Appl. No.: 10/948,558

(22) Filed: Sep. 23, 2004

(51) Int. Cl. A61B 5/05

(52) U.S. Cl. 600/415

(57) ABSTRACT

The present invention is directed to a system and method which allows for the mounting of a medical navigation system external to a patient. In one embodiment, displacement device is mounted around a patient's torso and serves to displace (e.g., distract or compress) the patient's skeleton in a manner such that the target site becomes fixed relative to a site on the displacement device. A navigation transmitter can then be mounted to the displacement device. The device has a quick disconnect that allows it to be removed without first releasing the displacement. Displacement can be controlled by pneumatic, hydraulic, electrical or by screw action. In one embodiment, the device can be used to position a patient on an operating table.

### TECHNICAL FIELD

This invention relates to medical procedures and more particularly to such procedures where it is necessary to track the location of tools and/or devices within a body being operated upon.

### BACKGROUND OF THE INVENTION

Many medical procedures, such as spinal operations, are now routinely performed in a minimally invasive manner, the desire being to reduce the trauma to the skin, soft tissue, and muscle as much as possible. A problem exists in such minimally invasive medical techniques in that when small skin incisions are made sight lines become non-existent and thus it is difficult to properly guide the surgical tools and/or implants when they are inside the body.

One method for such guidance is to continually take fluoroscope (X-ray) pictures as the procedure progresses. This is slow and suffers from problems due to harmful exposure for patients and medical personnel.

Another method uses fluoroscopy (X-ray) and electro magnetic electronic surgical navigation technology to keep track of the position of the tools and/or devices with respect to the spine. In such systems, it is important to establish a fixed relationship between a signal transmitter and the portion of the patient's anatomy being worked on. For discussion purposes, the structure(s) being worked on will be called the target site. This fixed relationship between the surgical tools and the electro magnetic transmitter must remain constant regardless of the motion of the target site. In order to accomplish such a fixed relationship, it is common practice to mount the transmitter on a bony surface adjacent to the target site. This adjacent surface is selected such that it has an anatomical connection to the target site and such that the adjacent site always moves in a constant relationship to that of the target site. In this manner, the transmitter at the adjacent site sends signals to the receiver unit mated to the surgical tools being used at the target site and those signals are reported back to a monitor so that the medical personnel can track the tool(s) in relationship to the target site. In essence then, the adjacent transmitter site provides a fixed point of reference for procedures with respect to the target site. A registration process of the transmitter to the anatomy may be performed in some instances. This includes first fixing the transmitter to a fixed landmark (most often bone). Second, an AP fluoroscopic image with the navigation calibration target attached to the image intensifier of the C-arm (Fluoroscope) is obtained. Next a lateral fluoroscopic image is taken. Now the patient's anatomy is registered to the transmitter and displayed on the surgical navigation monitor. This step is repeated every time the sensor (transmitter) is moved relative to the spinal anatomy. In order to navigate a surgical tool, the tool is first attached to a handle which has an imbedded receiver and then calibrated with the surgical navigation system. Each time the tool is changed, calibration must be repeated.

In a less optimal usage of this technology, the transmitter is not repositioned when the target site moves. In other words, the transmitter is not moved to the new target site, so that the transmitter and the target site are located on different vertebral bodies and therefore are not in the same fixed relationship that is optimal between the transmitter and target site. This suboptimal location contributes to the overall inaccuracy of the system.

Several problems arise in using such a system. One of these problems is that the transmitter must be implanted into the body. This implantation results in additional trauma to the body. A second problem is that for many procedures an adjacent bony surface upon which to mount the transmitter is not available. This unavailability could be because any such surface is too brittle, too small, too flexible, or perhaps lacking in depth for attachment of the transmitter. Also, the bony surface may not consist of hard bone, resulting in movement of the transmitter due to the soft nature of the fixation point. Also, such implanted transmitters are subject to being bumped by the surgeon and, from time to time, are in the line of sight. Also, for some procedures (for example, anterior approach) there may not be a sound anchor surface available. Also, in the event of an emergency situation these adjacent transmitters must be removed and this takes time when time is at a premium.

### BRIEF SUMMARY OF THE INVENTION

The present invention is directed to a system and method which allows for the mounting of a medical navigation system external to a patient. In one embodiment, an expandable device is non-invasively mounted around a patient's torso and serves to displace (e.g., distract or compress) the patient's skeleton in a manner such that the target site becomes fixed relative to a site on the displacement device. A navigation transmitter can then be mounted to the displacement device. The device has a quick disconnect that allows it to be removed without first removing the displacement. Displacement can be controlled by pneumatic, hydraulic, electrical, or by mechanical screw action, as examples. In one embodiment, the device can be used to position a patient on an operating table and can be fixed to prevent patient motion relative to the table.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows one embodiment of an externally mounted surgical navigation device;

FIG. 2 shows the externally mounted skeletal navigation device in the undistracted mode;

FIG. 3 shows the externally mounted skeletal navigation device after distraction;

FIG. 4 shows one embodiment of the navigation device being used during a medical procedure; and

FIG. 5 shows one embodiment of an externally mounted skeletal navigation device.

### DETAILED DESCRIPTION OF THE INVENTION

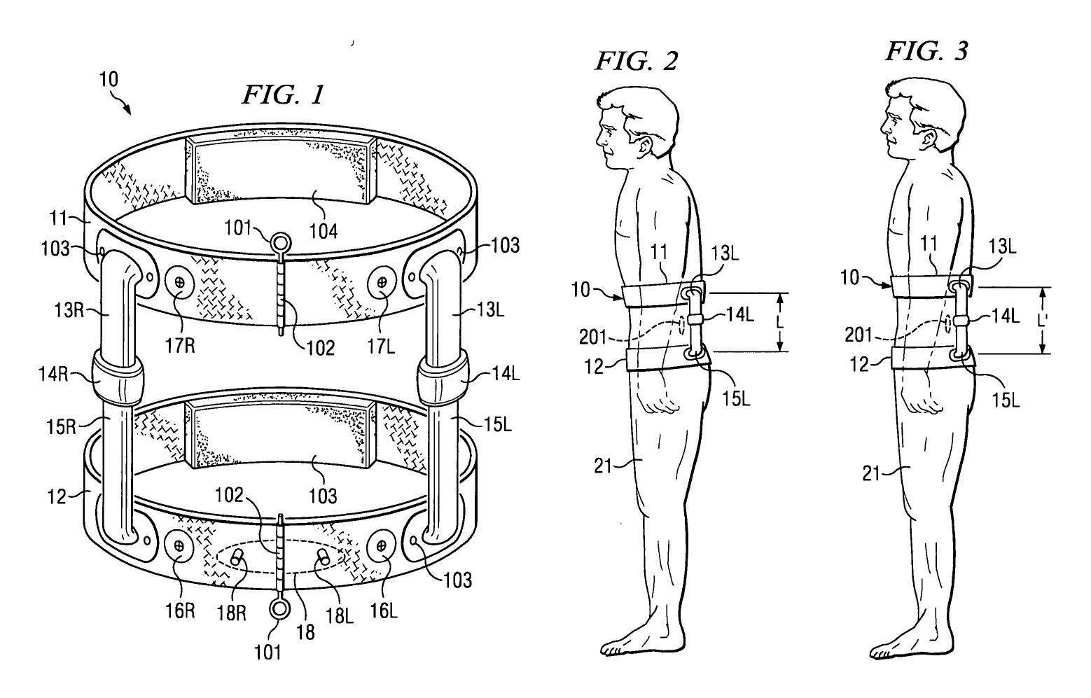

Turning now to FIG. 1, there is shown one embodiment of externally controlled surgical device 10. Device 10 can be used for surgical navigation, as well as for rigid positioning for a variety of surgical procedures, such as biopsy of soft tissue. The lower portion of device 12 is strapped around a portion of the patient below a target area, and band 11 is strapped around a portion of the patient on the other side of the target area. For example, when used in a spinal operation, the lower portion 12 may be strapped around the hips of the patient, and the upper band 11 may be strapped around the torso of the patient on a side of the target area opposite that of lower band 12. That is, in a spinal operation upper band 11 may be arranged, for example, above the vertebrae or disc to be operated on and lower band 12 may be arranged below the portion to be operated on which is the target site. Pads 103 and 104 can be placed all the way around the respective bands 11 and 12 or in certain areas. These pads can be permanently attached or can be added as needed during the tightening procedure. Each band portion 11 and 12 is tightened around a respective portion of the patient (e.g., around the patient's torso) by Velcro™ (not shown) or any other tightening/coupling method, such as for example a ratcheting strap, also not shown. The device may or may not include a gauge (such as gauge 510, FIG. 5) to measure how tight (tension) or how long (time) the device has been tightened down to an individual's body. Preferably, bands 11 and 12 would be made from an elastic fabric or from a rigid material (e.g., plastic or metal) that would conform to the individual's body. Loading points 17R and 17L, 16R, 16L could also be fabric (e.g., gel, foam, glue, pressure, surface coating, etc.) or a combination of a fabric fastened into a rigid cupped material on the inside of the bands to protect the torso and provide an anti-slip means where there is uniform load distribution on patient contact points, all without invading the patient's body. Note that the device can have several straps around the patient's body (arm, leg, etc.) or can have straps partially around the body, as long as there is sufficient grapping ability so that the device remains in contact with the body without slipping.

Lateral sides 13R and 13L and 15R, 15L interconnect bands 11 and 12 and are operable to displace band 11 relative to band 12 (e.g., to distract or compress the bands 11 and 12 relative to each other) under locking control of locks 14R, 14L. Note that in FIG. 2 lateral sides 13L, 15L are pre-positioned in this embodiment towards the posterior of the patient. Ideally the lateral sides would be located on the lateral side of the patient on either side of the spine but could be moved into position as desired depending upon the field of the operation and depending upon whether the physician is operating from the anterior/posterior or laterally to the target site. Also, in the embodiment shown, only two such lateral sides are shown, but any number could also be utilized, if desired. These lateral sides can be permanently mounted during manufacture or could be positioned to be mounted by a physician at the time of the operation. The adjustability of the lateral sides allows for this device to be customized for an individual's stature at the time of use. As shown in the example embodiment of FIG. 1, the lateral sides can be sewn into bands 11 and 12 or riveted or otherwise secured via holes 103.

For different patients, different adjustment mechanisms are possible. These adjustment mechanisms can be, for example, a lead screw, perhaps driven by a miter gear; a worm gear; a scissor jack, a hydraulic jack, pneumatic or electric piston, etc. One system could use an integrated plain bearing slide with a lead screw while another could use a pneumatic rear-mount air cylinder with either a ratchet lock, a spring return or a double-acting chamber. In such a system, when air is applied the piston expands. By connecting the lateral sides to work together, the patient's body is distracted by the application of air (or hydraulic fluid, or by hand cranking, etc.)

In the embodiment shown, locks 14R and 14L serve to control the lateral displacement of the lateral sides, such that once bands 11 and 12 are securely fastened to the torso of the patient the interconnecting lateral sides 13R, 13L, 15R, 15L are operated to controllably displace bands 11 and 12 relative to each other (e.g., to achieve distraction or compression). This displacement can be by, for example, turning locks 14R and 14L so that a screw extends the pressure upward to move band 11 away from band 12. Since band 11 then is attached to the torso of the patient above the vertebrae which is the target site, this procedure serves to distract the skeleton of the patient (for example, patient 21 in FIG. 2). When this distraction is far enough it effectively puts pressure on the skeleton so that a position on, for example, the hip bone which is in a relative position to band 12 is fixed with respect to any movement of the spine at the target site. Thus, device 10, when distracted as shown in FIG. 3, forces the spine to become, in effect, a rigid structure such that any movement of the body will maintain the target site in a fixed relationship to a specific position on band 12. Once the desired distraction force or distance is met, a locking mechanism keeps bands 11 and bands 12 from displacing in any direction.

In the embodiment shown, this specific position is recorded by navigation device 18R and/or 18L such as, for example, a navigation transmitter. While this example shows distraction, compression may be alternatively used in certain procedures (e.g., by turning 14R and 14L in an opposite direction to move band 11 toward band 12). Note that lateral sides can be designed to allow any type of patient adjustment, including flexion, extension, axial, or a combination thereof.

As discussed, FIGS. 2 and 3 show device 10 positioned around the torso of a patient 21 on either site of target side 201. In FIG. 2 the distance between bands 11 and 12 is length L. On FIG. 3 the bands 11 and 12 have been distracted by devices 13L and 13R such that the distance between the respective bands is the distance L prime (L′). The distraction force is expected to be approximately 30% of body weight which equates to approximately 60 lbs. (assuming a 200 lb. person). A distraction distance of about 3-4 inches to obtain the 60 lb. force distraction needed is anticipated.

Returning to FIG. 1, when the operation is complete or in the event of an emergency situation, device 10 can quickly be removed from the patient by, for example, pulling out quick release pins 101 which then allows device 10 to separate at 102. Note that this separation allows device 10 to be quickly removed from patient 21 without requiring the removal of the displacement (e.g., the distraction in the example of FIG. 3). Thus, even when a patient 21 is fully distracted and interconnection devices 13R and 13L are in the extended position, device 10 can be removed quickly.

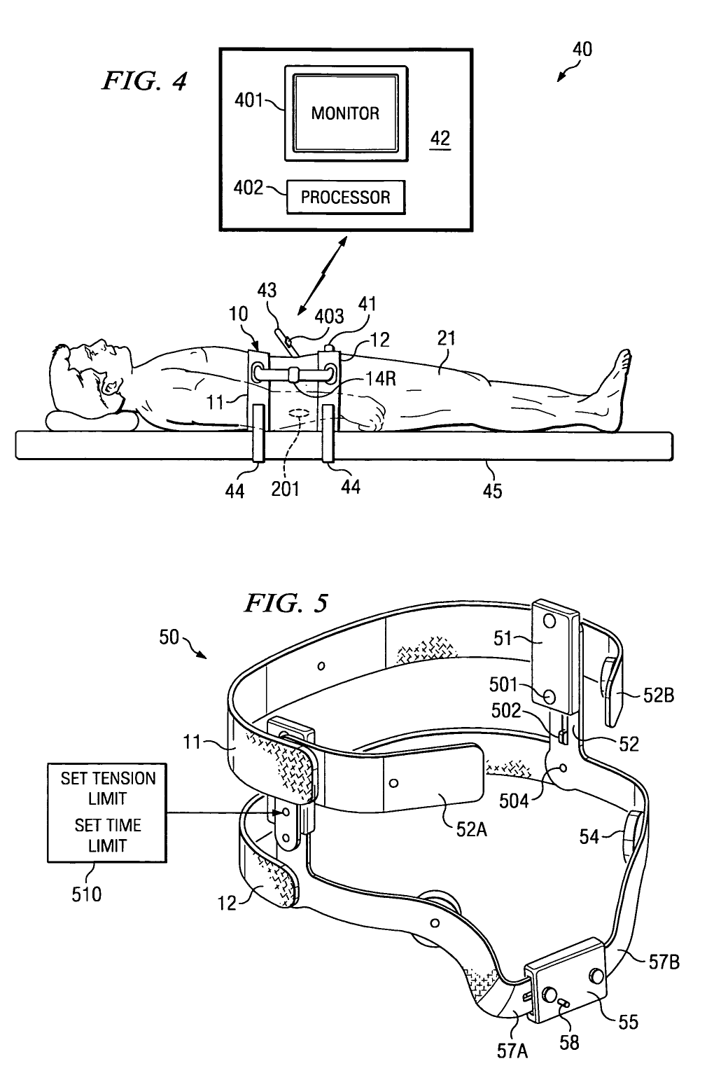

FIG. 4 shows one embodiment of the system and method of use of device 10 on patient 21. In this embodiment, patient 21 is fastened to operating table 45 by optional straps 44 which hold device 10 to the operating table for further stability of patient 21. As shown, transmitter 41 is mounted at position 18R and/or 18L of band 12 of device 10. Transmitter 41 is then in a fixed relationship with the target zone 201. Such that as the patient's body moves zone 201 remains in fixed relationship to transmitter 41. Tool 43, which may be used in a minimally invasive procedure in which the target area and/or tool are out of view of the physician, includes receiver 403 mounted thereon for sending signals directly to the CPU through a coaxial cable. As an example, published U.S. Patent Application No. 20030/0184285 describes an example navigation system, which includes a block diagram (FIG. 3) and text description of the process by which the signal is generated, read, and computed by the navigation system. While in this example the signals are transmitted via coaxial cable, in alternative implementations such signals may instead be transmitted wirelessly using any suitable wireless technology now known or later developed including without limitation Bluetooth, RF, etc. The transmitter 41 also sends a signal to a system through a coaxial cable, such as system 42 which includes processor 402 and monitor 401. System 42 then provides a visual display to the physician so that the physician can see the relative position between tool 43 and target site 201. When using this system in the well known fashion, then other tools and/or implanted devices can be properly positioned within a patient utilizing the tracking device as discussed herein.

Note that the distraction device discussed herein can be used to hold an instrument, retractor, attachment for a flexible arm, etc. during surgery to aid the surgeon during the procedure. Since the device is rigidly fixed to the patient and if an instrument, blade retractor system, or flexible positioning arm is then rigidly attached to the device, then as the patient moves the device moves in exact proportion and thus the tool also moves exactly proportional to the patient's movement.

FIG. 5 shows one embodiment 50 of a brace used to externally control skeletal navigation. In this embodiment, sides 51 and 52 interconnect bands 11 and 12. A locking mechanism, such as screw 501 rides in slot 502 of side portion 52. When the desired adjustment (distance between bands 11 and 12) is achieved, screw (or other locking device) is tightened to lock side portion 51 to side portion 52. Screw 503 (or other locking device) is used to fasten side portion 51 to band 11 while screw 504 (or other locking device) is used to fasten side portion 52 to band 12. Instead of screws, more permanent connections can be made, such as rivets, sewing, glue, etc. Note that a locking device need not be necessary if a piston (hydraulic, pneumatic) is used provided the pressure inside the piston is maintained. Also note that in a preferred embodiment, the adjusting mechanism will only go in one direction so that the device cannot be reused. To remove the expanded device from the patient the quick release mechanism is used.

In this embodiment, the inside surface of side portion 51 can be padded and pads 54 can be properly placed. Buckle portions 52A and 52B can be made to open and close as desired. This can be achieved through magnetic or electric field interaction, or through physical latching. Similarly, buckle portions 57A and 57B can be interlocked and signal transmitter (receiver) pad 55 with signal transmission connection (an antenna, if wireless) can be mounted to buckle 57A, 57B.

Device 510, which is optional, may or may not include a gauge to measure, for example, the distraction forces, distance, and/or time that the distraction has been applied. The gauge may include an audible alarm which indicates a distraction force has been applied for a specific amount of time or when an excessive distraction force has been reached. If desired, the tension and/or time may be set by the surgeon or during a pre-op procedure based upon parameters specific to the patient.

Note that while this procedure has been discussed with respect to a patient's spine, it can be used with respect to any two structures in the skeleton, which when distracted will remain rigid with respect to each other without regard to the movement of the skeleton. As discussed herein, system 40, shown with respect to FIG. 4 and with respect to device 10, can be used for several operations of the skeleton. For example, spinal fusion, Artificial Disc Replacement, MIS pedicle screw insertion are but a few of the procedures that device 10 in the system and method described herein can be used. Also, as discussed above, since the device holds a patient's body rigid, it can be used for other procedures, such as, for example, to perform a biopsy of a certain segment of the body that would otherwise not be rigid.

In operation, when placement of the lower and upper collars 111 and 12 are used it would be helpful if they are placed on bony landmarks that are known, such as, for example, pelvis and/or rib cage. The lifting mechanism of FIG. 10 is utilized to axially distract the spine when bands 11 and 12 are tightened with respect to the torso. It is important that they do not slip and there is a uniform load distribution on contact. In this regard, pads can be used, but also glue or other surface coatings gels can be utilized to help insure that device 10 is locked solidly to the patient's skin. Note also that lateral sides 13R, 15R and 13L, 15L can be kinematic, pneumatic, mechanical, electrical or hydraulic and could have screws or levers to displace the upper band 12 with respect to the lower band 11. Note also that the device is not limited to use in the lumbar area but could, for example, be used in the cervical area and even for thoracic procedures.

While the device shown herein is designed primarily for use in spinal distraction for the purpose of performing medical procedures internal to the body, the device could also be used to correct scoliosis by providing distraction forces which are differential, i.e., greater in one quadrant for deformity correction of the spine. This could be a situation where the device is worn for a period of time by a patient or for correction of a spine temporarily while a physician performs an operation to implant a fusion device (screws or rods), or to correct other deformities internal to the patient. Further, in some procedures the device 10 may not only be worn during an operation, but may also be worn for a period following the operation to, for example, monitor the position of an implant that includes a transmitter similar to that described above for receiver 403 of tool 43, or simply to stabilize the patient during the initial healing process.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A medical device comprising:
first and second supports for temporary attachment to first and second spaced apart portions of a patient's body; and
at least one displacement device for adjustably displacing said first and second supports so as to effectively prevent relative movement of the anatomy between said first and second supports.

2. The device of claim 1 wherein one of said supports is adapted for positioning a medical instrument thereon.

3. The device of claim 2 wherein said medical instrument is a tracking transmitter for exchanging signals with a medical tool within said patient.

4. The device of claim 1 wherein said first and second supports are part of a common device mounted externally on said patient.

5. The device of claim 1 wherein said at least one displacement device is rigidly attached to at least one of said supports.

6. The device of claim 1 wherein said supports fit around at least a part of the patient's body.

7. The device of claim 1 wherein said at least one displacement device is operable to distract said first and second supports.

8. The device of claim 1 wherein one of said supports transfers load onto the hip of said patient and wherein when said displacement device adjustably displaces said supports to a position where said relative movement of said bony structure has been prevented so that said hip becomes a fixed reference point with respect to a given portion of said anatomy.

9. The device of claim 1 further comprising:
at least one quick release mechanism for allowing said navigation system to be removed from a patient while said displacement device is adjustably displaced.

10. The device of claim 1 further comprising:
at least one gauge for determining limits on said displacement.

11. The device of claim 10 wherein said limits are selected from the list of time, tension, compression.

12. A method for providing a relatively fixed location for use in a medical navigation system; said method comprising:
immobilizing a target area within a patient's body by distracting a portion of the body surrounding said target area, said distraction being accomplished by temporarily non-invasively attaching first and second supports to first and second exterior portions of said patient and wherein said fixed location is associated with at least one of said supports.

13. The method of claim 12 further comprising:
exerting force to said first and second structures, said force acting to push said structures apart from each other.

14. The method of claim 12 further comprising:
attaching a communicating device to said fixed location such that the communication device maintains a fixed relationship with said target area regardless of the movement of said patient's body.

15. The method of claim 12 wherein said target area is part of said patient's skeleton and lies within said patient's body between said supports, and wherein said exerted force is sufficient to effectively prevent relative movement between the bones of said skeleton.

16. The method of claim 12 further comprising:
removing said distraction device by activating a quick release mechanism.

17. The method of claim 12 further comprising:
providing an indication of certain distraction parameters.

18. The method of claim 17 further comprising:
sounding an alarm when certain of said distraction parameters are achieved.

19. A medical navigation system comprising:
a first communication device for exchanging signals from fixed positions relative to a patient's body;
a second communication device for exchanging said signals from at least one medical device within a patient's body;
a processor for processing exchanged signals and for providing visual images of at least a portion of each said device as each said device moves within said body;
first and second supports for temporary attachment to first and second spaced apart portions of said patient's body; and
at least one displacement device for adjustably displacing said first and second supports so as to effectively prevent relative movement of the bony structures between said first and second supports and wherein a rigid part of said displacement device is adapted for positioning said first communication device.

20. The system of claim 19 wherein said signals are wireless.

21. The medical navigation system of claim 19 comprising:
a displacement device for separating said supports so as to establish a fixed position relative to a target structure within said body between said supports and said one support having said first communication device positioned in association therewith.

22. The medical navigation system of claim 21 wherein said supports are mounted at least partially around the torso of said patient.

23. The medical navigation system of claim 22 wherein said system further comprises:
at least one quick release device for allowing said supports to be removed without first releasing said adjustable displacement.

24. The medical navigation system of claim 21 wherein said system further comprises means for presenting said displacement device from being reused.

25. The medical navigation system of claim 19 wherein said adjustable displacement is controlled by at least one of the following:
hydraulic, pneumatic, electric, scissors, pulleys, gears, lead screw.

26. The medical navigation system of claim 19 wherein said at least one displacement device is operable to selectively distract said first and second supports.

27. The medical navigation system of claim 19 wherein said at least one displacement device is operable to selectively compress said first and second support.

28. A method for positioning a medical device on a patient, said method comprising:
placing first and second portions of a displacement device around first and second portions of a patient's body, said first and second portions on either side of a medical target site;
displacing said first and second portions relative to each other in an amount such that said target site comes into temporary fixed relationship with at least one of said device portions; and
placing a navigation device on at least one of said device portions.

29. The method of claim 28 further comprising:
performing a medical procedure at said target site using said placed navigation device for tool positioning assistance.

30. The method of claim 28 further comprising:
removing said device portions from said patient's body without first releasing said displacing of said first and second portions.

31. The method of claim 28 further comprising:
clamping said first and second device portions to an operating platform.

32. The method of claim 28 wherein said displacing comprises distracting said first and second portions relative to each other.

33. The method of claim 28 wherein said displacing comprises compressing said first and second portions relative to each other.

34. The method of claim 28 further comprising:
providing an indication of certain distraction parameters.

35. A medical procedure and navigation system comprising:
means for externally displacing a patient's skeletal anatomy on either side of a target site; and
means for mounting a navigation device on said displacing means, said mounted navigation device in fixed relationship with patient structures at said target site when said external displacement of said patient has been achieved.

36. The medical procedure and navigation system of claim 35 wherein said displacing means comprising:
interconnecting at least a pair of torso surrounding bands.

37. The medical procedure and navigation system of claim 36 wherein said interconnecting means is selected from the list of: pneumatic, hydraulic, electrical, mechanical systems.

38. The medical procedure and navigation system of claim 36 wherein said interconnecting means is operable to perform at least one of the following:
distraction of said pair of anatomy stabilizing bands relative to each other, and
compression of said pair of anatomy stabilizing bands relative to each other.
